(19) United States
(12) Patent Application Publication
(10) Pub. No.: US 2010/0063466 A1
(43) Pub. Date: Mar. 11, 2010

(54) ABSORBENT ARTICLE

(75) Inventors: Ulrika Husmark, Molnlycke (SE); Ingrid Gustafson, Asa (SE)

Correspondence Address: DRINKER BIDDLE & REATH (DC), 1500 K STREET, N.W., SUITE 1100, WASHINGTON, DC, 20005-1209, US

(73) Assignee: SCA Hygiene Products AB, Goteborg (SE)

(21) Appl. No.: 12/514,570

(22) PCT Filed: Nov. 17, 2006
(86) PCT No.: PCT/SE2006/050488

§ 371 Date: May 12, 2009

(51) Int. Cl. A61L 15/46

(52) U.S. Cl. 604/360

(57) ABSTRACT

An absorbent article, such as a diaper, diaper pants, sanitary towel or incontinence protector, with a vapor-permeable backing layer, including a liquid-permeable cover layer intended to be directed toward the user during use, a liquid-impermeable but vapor-permeable backing layer intended to be directed away from the user during use, and an absorbent core between the cover layer and the backing layer. The article has a longitudinal direction, a transverse direction, two substantially longitudinal side edges, a substantially transverse front edge, a substantially transverse rear edge, a substantially longitudinal midline, and a front part and a rear part on each side of a center line. The midline and center line intersect one another at a point of intersection. The absorbent article includes at least one monovalent salt of the form X⁺Y⁻ in a quantity of 1-75% by weight calculated on the basis of the weight of the core.

### CROSS-REFERENCE TO PRIOR APPLICATION

This application is a §371 National Stage Application of PCT International Application No. PCT/SE2006/050488 filed Nov. 17, 2006.

### FIELD OF THE INVENTION

The present invention relates to an absorbent article, such as a diaper, diaper pants, sanitary towel or incontinence protector, with a vapour-permeable backing layer, comprising at least one monovalent salt of the form X⁺Y⁻.

### BACKGROUND OF THE INVENTION

Microorganisms commonly appear in absorbent articles during use. Microorganisms are introduced into absorbent articles via body fluids, such as urine or menstrual fluid, or by contact with the skin. The growth of bacteria in absorbent articles may be considerable during the time these articles are in use. With a high number of microorganisms there is an increased risk of urinary tract infections and skin irritation, and odors are also to a large extent associated with the presence of bacteria.

The bacteria are often ones that commonly occur in the individual. There is normally an ecological balance between different microorganisms on the skin and mucous membranes, and the normal microbial flora is important for ensuring that undesired microorganisms do not have the chance to start growing. Sometimes this balance can be disturbed, such that potential pathogenic microorganisms may establish themselves, grow and then give rise to infections, for example in connection with medication, poor hygiene, poor immune defense, excessive hygiene, skin changes, changes in the mucous membranes, and long-term use of absorbent articles.

The use of absorbent articles over a long period of time without regular changing or with poor hygiene, combined with an impaired immune defense, can increase the growth and spread of microorganisms. Moreover, tight-fitting clothes and underwear can increase the risk of growth of microorganisms. With an increased number of undesired microorganisms in an individual or in an absorbent article, there is an increased risk of microbial imbalance and of infections.

A natural part of the prevention of urogenital infections lies in improved personal hygiene. However, it may not be appropriate to wash the genital area with strong soap or bactericidal agents, and it may therefore be difficult for the individual to reduce the risk of infection to a suitable level using conventional means. Excessively thorough washing with strong cleaning agents also disturbs the normal flora of desired bacteria that help protect us against the undesired bacteria. It can also sometimes be difficult to change absorbent articles. Long-term use can also increase the risk of infections if microorganisms continue to grow within the article. Occlusion and temperature favor the growth of microorganisms in the article and on the user's skin and mucous membranes.

Resulting infections have traditionally been treated with conventional antibiotics. However, repeated treatment with antibiotics can lead to the development of resistant bacterial strains, which can make future treatment of infections difficult. A further problem with antibiotic treatment is that many individuals are hypersensitive to antibiotics. Moreover, antibiotics may be damaging to the skin and to the vaginal flora in women, with the result that the infection may reappear. The use of antibiotics can also have the disadvantage of disturbing and destroying the bacteria in the normal flora.

Previous attempts have been made to reduce the growth of microorganisms and bacteria in absorbent articles by addition of bacteriostatic agents and lowering of the pH.

It has also been proposed to use lactic acid bacteria on account of their inhibiting effect on pathogens. The use of lactic acid bacteria has been shown to reduce the occurrence of infection on both skin and mucous membranes.

WO 2004/105822 describes the use of absorbent articles provided with bacteria that produce lactic acid. The bacteria cells are transferred to the user's skin and reduce the risk of microbial infection in the user's urogenital region and on the user's skin.

EP 1 032 434 B1 describes an absorbent article that contains lactic acid bacteria. The bacteria are arranged to be transferred to the user's skin in order to worsen the conditions for undesired microorganisms.

EP 510 619 cites a number of agents that prevent the growth of bacteria, for example chlorhexidine, quaternary ammonium compounds, copper salts, chelating agents, parabens, chitin and pH buffers.

Several examples of antimicrobial agents are cited in US 2004/0180093, for example silver compounds, copper compounds, and zinc compounds contained in a polymer composition. Silver, copper and zinc ions have antibacterial properties.

U.S. Pat. No. 4,883,478 describes the preparation of an absorbent composition containing saccharide. Monosaccharide and/or disaccharide is mixed with superabsorbent to give a homogeneous paste. The absorbent material can be used in dressings, for example. The composition is especially advantageous for wound treatment, since sugar favors the healing of wounds. Sugar also lowers the water activity, and this prevents growth of microorganisms. However, sugar has to be concentrated in order to function as a preservative. When diluted with urine, for example, it functions as a nutrient substance.

Lowering the pH can also inhibit the growth/activity of bacteria. This can be done, for example, with acidic superabsorbent polymers, acidic pulp, the addition of acids, etc.

Absorbent articles in some cases comprise liquid-impermeable but vapor-permeable backing layers. Such backing layers allow gases to pass through, such as air and water vapour, but are impervious to liquid such as urine and menstrual fluid. In this way, the moisture content against the user's skin during use is lowered, and the product is felt to be airier. Liquid can come directly from the user's skin through seating, or it can come from excreted body fluid taken up in the absorbent core. The reduction in moisture through use of vapor-permeable layers has not been sufficient to reduce the growth of microorganisms. Foul odors can also penetrate through a vapor-permeable layer.

Lactic acid bacteria in absorbent articles require special protection during storage, and a more stable solution is needed to the problem. It would also be desirable to find a solution that is more environmentally friendly and simpler than the earlier solutions outlined above.

The undesired presence of microorganisms in absorbent articles, even at a low concentration, can create possibilities for pathogenic microorganisms to increase in numbers in certain situations. The risk of foul odors in absorbent articles then increases, since undesired bacteria often lead to the occurrence of bad odors. There is therefore a need to prevent the occurrence and growth of microorganisms in absorbent articles. The present invention is aimed at solving these problems.

### SUMMARY

The object of the present invention is to provide an absorbent article with improved hygiene which reduces the risk of infections and of foul odors by limiting the growth and activity of undesired microorganisms.

This can be achieved, according to an exemplary embodiment of the present invention, by an absorbent article, such as a diaper, diaper pants, sanitary towel or incontinence protector, with vapor-permeable backing layer, comprising at least one monovalent salt of the form X⁺Y″ in a quantity of 1-75% by weight (% dry) calculated on the basis of the weight of the core.

### BRIEF DESCRIPTION OF THE FIGURES

FIG. 1 shows an absorbent article according to the invention in the form of a sanitary towel.

FIG. 2 shows an absorbent article according to the invention in the form of an incontinence protector intended for men.

FIG. 3 shows an absorbent article according to the invention in the form of a sanitary towel, the crotch area being illustrated.

FIG. 4 is a diagram showing the growth of bacteria at different salt additions.

FIG. 5 shows ammonia production at different salt additions.

FIG. 6 is a graph showing the rising salt concentration in combination with a vapor-permeable layer.

FIG. 7 shows water activity in 3% strength salt solutions.

FIG. 8 shows the water activity at different dilutions of salts in Elga-H₂O.

### DEFINITIONS

The inventors define two different ways of specifying the salt content in a product. The first is % dry. % Dry describes the quantity of salt in relation to the weight of the core, as shown below. This description is used by us for the dry product, i.e. when the product is produced and before it is used.

% dry=Ms/(Ms+Mk)

Ms=mass of the added salt

Mk=weight of the core, e.g. weight of SAP+weight of pulp+any other ingredients in the core.

The inventors also define a salt content in the wet product, namely % wet. % Wet describes the quantity of added salt (% dry) that dissolves in the liquid when the product is used. The quantity of liquid varies depending on the user's situation, and the liquid can be synthetic test liquid or real body fluids such as urine, menstrual fluid, etc.

% wet=Ms/Mv

Ms=mass of the added salt

Mv=mass of the liquid

The resulting salt content, and thus also the water activity in the wet product, is an important factor for whether and to what extent the bacterial growth is inhibited and hygiene thus improved. Therefore, % wet is an important parameter to use in describing the effect of added salt (% dry). When the product is wetted with urine, test liquid, etc., salt is added with the inherent salt content of the liquids, but this has not been calculated into the specification of % wet.

Water activity is a measure of the quantity of free water in a system and is an important factor for whether microorganisms can grow or not. The definition for water activity is: A_{W}=P_{S}/P₀ where P_{S} is the water vapor's partial pressure over the specimen at a certain temperature, and P₀ is the water vapor's partial pressure over pure water—at the same temperature. The water activity is a dimensionless number between 0 and 1, where the water activity for pure water is 1, and, when the water activity is 0, there is no free water.

“Pure salt” is understood as at least 99% by weight of salt.

“Wet zone” is to be understood as the zone of the absorbent article that receives the body fluid. This zone is often placed at the center of the absorbent article. If the article has a front part, a rear part and a crotch part, the crotch part is often the part of the absorbent article that first receives body fluid. In the case of an incontinence protector designed for men, the wet zone is essentially the part of the article that receives urine from the penis, and this is usually the central part of the article. Since an absorbent article can be placed differently on the user's body and can differ from case to case, reference is also made to an “intended wet zone”. This is the zone that is intended to function as wet zone during use of the absorbent article, and this is of course also the central part, as mentioned above.

### DETAILED DESCRIPTION OF PREFERRED EMBODIMENTS

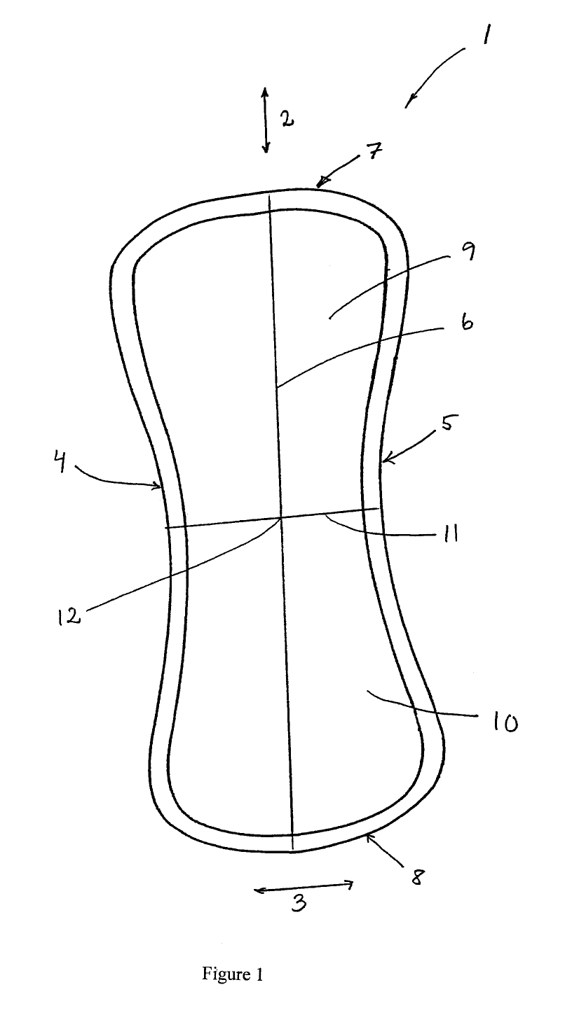

The present invention relates to an absorbent article 1, such as a diaper, diaper pants, sanitary towel or incontinence protector, with a vapor-permeable backing layer, FIG. 1 showing a sanitary towel comprising a liquid-permeable cover layer intended to be directed toward the user during use, a liquid-impermeable but vapor-permeable backing layer intended to be directed away from the user during use, and an absorbent core between the cover layer and the backing layer, said article having a longitudinal direction 2, a transverse direction 3, two substantially longitudinal side edges 4, 5, a substantially transverse front edge 7, a substantially transverse rear edge 8, a substantially longitudinal midline 6, a front part 9 and a rear part 10 on each side of a substantially center line 11, said midline 6 and center line 11 intersecting one another at a point of intersection 12, and the absorbent article comprises at least one monovalent salt of the form X⁺Y⁻ in a quantity of 1-75% by weight (% dry) calculated on the basis of the weight of the core. When liquid such as urine is added to the absorbent article, the water activity in the absorbent article increases. When the salt is present in an absorbent article, it will lead to a lowering of the water activity in the absorbent article when the liquid has come into contact with the salt. The article preferably contains 5-55% by weight (% dry) of salt calculated on the basis of the weight of the core. The transverse center line 11 is preferably placed substantially at the midway point of the length of the article.

During use, water vapor is driven out through the vapor-permeable/breathable backing layer. The driving force of the water vapor out through the backing layer will of course be affected by the environment on the outside of the backing layer. Water vapor on the inside is more easily conveyed outward if the moisture content on the outside of the article is low. When water vapor is conveyed out through the layer, the result is that liquid disappears from the absorbent article, but the salt remains in the article. This leads to a rise in the salt concentration in the excreted liquid remaining in the article, and this in turn leads to a further drop in the water activity, which further reduces the bacterial growth. When the bacterial growth decrease, there is the further advantage that undesired odors are inhibited. The disadvantage of a vapor-permeable backing layer allowing undesired odors to pass through can thus be eliminated. A drop in temperature is also achieved as the vapor is driven off, and the bacteria will then grow more slowly. This provides better comfort and hygiene.

Using salt in an absorbent article is simple and inexpensive. It may also be more environmentally friendly than using other antimicrobial agents, it can be good for the skin, and it does not make bacterial strains resistant. The salt thus lowers the water activity in a wet absorbent article. A wet absorbent article is also to be understood as an article that is moist with excreted material from the user's genital area or skin. The salt coordinates water and thereby reduces water activity, which inhibits microbial growth during use. This is an advantage in hygiene terms, since it, for example, reduces the risk of infections. The reduced microbial growth also functions as an odor-inhibitor, since microorganisms, such as bacteria for example, produce malodorous substances, for example ammonia. The quantity of ammonia produced is decreased by virtue of the invention.

The combination with a vapor-permeable backing layer is thus extremely effective, since bad odors are not generated to such a great extent in the article, and there is therefore a lesser quantity of bad odors that can pass through the backing layer. This effect is intensified as the water activity is lowered by way of the vapor-permeable backing layer.

The quantity of free water, the water activity, is a very important parameter that determines whether bacteria, yeast or mold can grow in various environments. In absorbent articles, it is primarily bacteria that grow. Bacteria are the group of microorganisms that is first inhibited when the water activity decreases.

The backing layer is liquid-impermeable, but vapor-permeable, in order to allow water vapor to pass through from the article. This is especially advantageous if it is a large article, since it provides a larger surface area for escape of vapor.

X⁺ is chosen from among Na⁺, K⁺, [[NH₄⁺]] NH₄⁺, and Y⁻ is chosen from among C₂H₃O₂⁻ and C₃H₅O₂⁻. The monovalent salts can be chosen from among NaCl, NaC₂H₃O₂ (Na acetate), NaC₃H₅O₂ (Na propionate), KCl, KC₂H₃O₂ (K acetate), KC₃H_{S}O₂ (K propionate), NH₄Cl, NH₄C₂H₃O₂ and NH₄C₃H₅O₂. These monovalent salts perform well in reducing the water activity.

Polyvalent ions also work, but polyvalent positive ions can have a negative effect on the superabsorbent polymer. By ion bonding, polyvalent positive ions can interact with the superabsorbent polymer network and negatively affect the swelling capacity and thus its absorption, more negatively than do the monovalent salts.

The salt is preferably distributed within a central area around the point of intersection 12. This area is regarded as a wet zone, since it is the area that urine and excreted body fluids will first arrive at on the absorbent article. The salt is thus preferably distributed in the intended wet zone.

It is advantageous to place the salt in and around the area round the wet zone, since the salt needs to be in contact with the liquid that is applied to the absorbent article in order to be able to reduce the water activity to any extent. The size of the wet zone depends on which absorbent article is intended, how much liquid the user excretes, etc. A diaper for example has a larger wet zone than a sanitary towel. Moreover, the wet zone for a relatively large incontinence protector will be larger than for a small incontinence protector. In the wet zone, the salt is dissolved in the liquid and is then transported with the liquid to other parts of the article.

The area within which the salt is distributed extends at least 1.5 cm from the center line 11 in the longitudinal direction 2 and at least 1.5 cm from the midline 6 in the transverse direction 3. The area extends in both directions from the center line and the midline. This is an area which to a large degree will receive liquid from the user when the article is applied. To make the best use of it, the salt should be placed in this area. The salt can also be placed in an area that covers the whole of the absorbent article. It will then not come into contact with liquid everywhere, but it may be simpler to add the salt without restricting the positioning thereof.

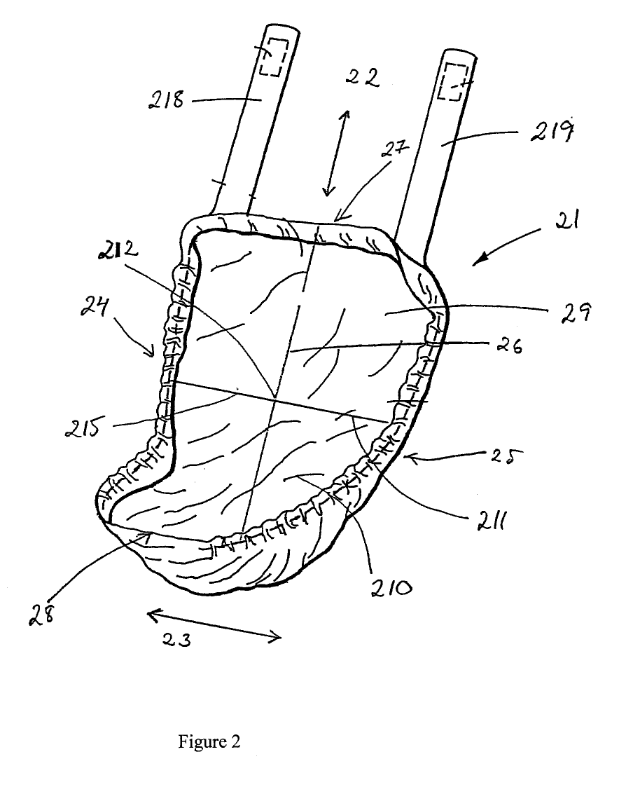

According to one embodiment of the present invention, the absorbent article is an incontinence protector intended for men, and it is intended to be placed such that it covers the male member. An incontinence protector 21 for men is shown in FIG. 2. Like the absorbent article in FIG. 1, the article in the form of an incontinence protector for men in FIG. 2 has a liquid-permeable cover layer intended to be directed toward the user during use, a liquid-impermeable backing layer intended to be directed away from the user during use, and an absorbent core between the cover layer and the backing layer, said article having a longitudinal direction 22, a transverse direction 23, two substantially longitudinal side edges 24, 25, a substantially transverse front edge 27, a substantially transverse rear edge 28, a substantially longitudinal midline 26, a front part 29 and a rear part 210 on each side of a substantially transverse center line 211. The midline 26 and center line 211 intersect one another at a point of intersection 212. The incontinence protector also has fastening arrangements 218, 219. The salt is distributed within a central area around the point of intersection 212. In the same way as above, the salt is distributed within an area that extends at least 1.5 cm from the center line 211 in the longitudinal direction 22 and at least 1.5 cm from the midline 26 in the transverse direction 23.

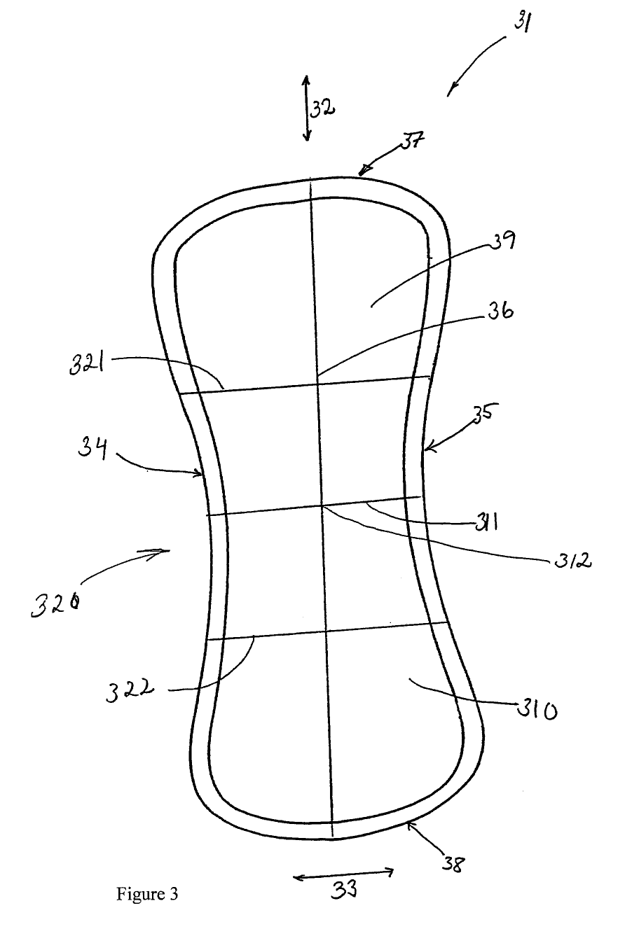

Some absorbent articles also comprise a crotch part. Such an article is shown in FIG. 1 and in FIG. 3. The crotch part is not shown in FIG. 1, but is illustrated in the absorbent article 31 shown in FIG. 3. Like the absorbent article in FIG. 1, the article has a liquid-permeable cover layer intended to be directed toward the user during use, a liquid-impermeable backing layer intended to be directed away from the user during use, and an absorbent core between the cover layer and the backing layer, said article having a longitudinal direction 32, a transverse direction 33, two substantially longitudinal side edges 34, 35, a substantially transverse front edge 37, a substantially transverse rear edge 38, a substantially longitudinal midline 36, a front part 39 and a rear part 310 on each side of a substantially transverse center line 311. The midline 36 and center line 311 intersect one another at a point of intersection 312. The crotch part 320 is placed substantially at the center of the absorbent article. The center line 311 passes through the crotch part 320, which has a length of about 7-12 cm. The crotch part is delimited in the figure by a front, substantially transverse border 321 and by a rear, substantially transverse border 322. Absorbent articles with a crotch part are, for example, sanitary towels, diapers and incontinence protectors, but not incontinence protectors intended for men, where the protection is intended to be positioned such that it essentially covers the male member. The crotch part is the area that receives excreted body fluids and can define a wet zone, and salt is distributed preferably in a crotch part of an absorbent article.

In some cases it is advantageous if a larger area of the article is wet. For example, for products having the ability to take up a lot of liquid, it is expedient if the liquid is distributed over a large area of the article. A larger amount of vapor will then escape through the vapor-permeable backing layer. It can then be advantageous to have a spreader layer in the absorbent article, which layer spreads the liquid in the directions outward from the wet zone. The greater the area within which the liquid is spread, the better the escape of vapor through the vapor-permeable backing layer. The absorbent article thus preferably comprises a spreader layer.

A spreader layer can comprise a material having a capillary force greater than the adjacent material from which it will receive liquid. The spreader layer will also have the ability to quickly suck liquid by a capillary effect along the plane of the layer. A spreader layer can be produced by the wet-laying principle, by which means a dry paper layer with wet strength is obtained. It is also possible to produce a spreader layer using the air-laying principle. For a dry-laid structure to exhibit good strength, binders such as glue or melt fibers should be admixed. The spreader layer can include a mixture of one or more different types of cellulose fibers, non-cellulose fibers, such as wool, silk, or synthetic fibers. Compressed chemical pulp can be used, as can nonwoven material, for example spunlace material, meltblown material and the like.

The spreader layer can be arranged between the cover layer and the absorbent core or between the absorbent core and the backing layer. Moreover, the spreader layer can be arranged within the absorbent core, for example between two absorption sheets.

For some products, however, it is not advantageous to include a spreader layer. This applies, for example, to articles intended for a small quantity of liquid.

The salt does not need to be distributed in the whole area that the liquid will cover. The liquid dissolves the salt and will then transport the salt with it.

The salt can also be placed in an area that covers the whole absorbent article. It will then not come into contact with the liquid everywhere, but it can be simpler to add the salt without restricting the positioning thereof.

The salt can be distributed within the absorbent core. This is the place where liquid is collected, and it is important that the salt comes into contact with the liquid collected in the absorbent core.

The salt can also be distributed on top of the absorbent core. There, it comes directly into contact with incoming liquid as soon as the liquid has passed through the cover layer and any other layers arranged between the cover layer and the absorbent core. The salt is dissolved and can then be transported down into the absorbent core.

The absorbent article comprises a vapor-permeable backing layer which can have a vapor permeability of at least 500 g/m² and 24 h, measured at 23° C. according to the ASTM-6701 MDCON method. Such vapor permeability is needed so that vapor will be able to pass outward through the backing layer in a satisfactory way and so that it can give an effect on the salt content.

The salt can have a particle size of 50 to 1500 μm. The salt can be in the form of grains, and it is the diameter of the grains that is stated. Since the salt grains can be angular shaped and can be irregular in shape, the diameter concerned is an average diameter. The salt can also be in the form of some other particle shape, such as flakes. The size of the flakes refers to the extending size of the flakes, i.e. the width of the flakes, and can be up to 1 cm. The flakes can have a thickness of up to about 5 mm. The size can depend on which article they are placed in, where in the article the salt is placed, and whether a rapid or slow dissolution of the salt is wanted. If the salt is placed on top of the absorbent core, it may be expedient to have relatively small grains. Liquid quickly comes into contact with the salt, and the liquid will pass quite quickly down into the absorbent core and is probably not in contact with the solid salt for any long time. If the salt is placed under the absorbent core, the liquid has already passed through the core and will not be transported farther downward in the article. Small grains or particles may be expedient if the salt does not come into contact with as much liquid (since the liquid does not pass through the salt) as it does when the salt is placed within or on top of the core. At the same time, large grains may also be suitable, since the liquid and the salt can be in contact for a relatively long time.

As has already been mentioned, the salt is preferably placed in a central area around the point of intersection of the midline and the center line, regardless of which layer the salt is placed in or on or under. In addition, the absorbent core can be divided into several absorbent sheets and the salt can be placed between these sheets. Moreover, the salt can be placed in one of several sheets or in all the sheets of an absorbent core. Further layers, such as an airlaid layer, arranged between cover layer and absorbent core, can also include the salt. Moreover, a layer can be arranged under the absorbent core, between the core and the backing layer where the salt is placed.

The salt can be added as pure salt, i.e. with a purity of 99%. It therefore does not really contain any additives. The salt is an inexpensive constituent of absorbent articles, and it is also easy to apply it to the article. The salt can also be applied by spraying on of a salt solution, or a layer of the absorbent article can be impregnated with or immersed in a salt solution, and the salt solution is then allowed to evaporate such that the solvent in the form of water, for example, is dried off from the article. The salt will then be present in the form of salt crystals in the absorbent article. The salt is preferably in pure form, i.e. with a purity of 99% by weight of salt.

The salt can be applied in the form of particles and mixed in during forming of the absorbent core. The salt grains can of course also be applied in distinct layers under the core, inside the core or on top of the core. If the salt is dissolved in liquid or suspended in a liquid, the salt solution or the salt suspension is applied to the absorbent core by spraying. The spraying is followed by a drying step. It is advantageous if the salt solution/salt dispersion/salt suspension is applied already by the manufacturer, since the spraying step can be avoided during the preparation of the article. Salt solution can be sprayed onto one or both sides of an absorbent core.

The solvent used for the salt can be water. A volatile organic solvent such as ethanol can be used as dispersant for the salt, or a mixture of a water-miscible organic solvent such as ethanol. The ethanol will function as a carrier for the salt.

The undesired bacteria whose growth is prevented are very sensitive to a decreased water activity. E. coli and Proteus reduce their growth even at a water activity of 0.98 and have great difficulty in growing below a water activity of 0.95.

There are several contributory factors for the reduced water activity in an absorbent article. Urine has a salt content of about 1%, which also gives a decrease in the water activity. The salt content in urine varies greatly from person to person and from case to case (depending, among other things, on how much has been drunk). The water activity will then depend on the amount of added salt, on which salt has been added, on what quantity of urine is conveyed to the article, and on the urine's inherent salt content.

The salt can be added in a quantity such that the water activity in the absorbent article is under 0.98. A lower limit for the quantity of salt that has to be added is, for example, 10-30% by weight calculated on the basis of the weight of the core and an incontinence product with a high load. Even such a low content as 1% by weight can in some products give a water activity of under 0.98. Higher salt contents and, consequently, lower water activity provide better inhibition of bacterial growth. The salt content of urine will also contribute to the decrease in water activity. An upper limit of 75% by weight (% dry) is directed primarily for reasons of production engineering.

It has been found that a salt content of about 3% by weight (% wet) in synthetic urine is very advantageous for decreasing the water activity, for reducing the production of ammonia and for inhibiting bacterial growth in absorbent articles. Various tests have been carried out for 1, 2 and 3% by weight (% wet) of added salt in synthetic urine as are described below under the examples. It is therefore advantageous, in an absorbent article that is wet, to have a salt concentration of about 3% by weight (% wet) of added salt to synthetic urine. 1 and 2% by weight (% wet) of added salt also function well.

An absorbent article has a maximum absorption capacity that differs for different products. For the quantity of added salt to be 3%, or 2 or 1% (% wet), when wetted during use of an absorbent article, different quantities of salt therefore have to be added, depending on which product is involved. The products have different weights, different weights of the core, and different maximum absorption capacity. To reach 3% by weight (% wet) of salt in the liquid in an absorbent article, the maximum absorption capacity has been calculated, i.e. the maximum weight of liquid that an absorbent article is able to absorb, and from this it is possible to calculate how much salt needs to be added to the absorbent article to reach a salt content of about 3% by weight (% wet). An absorbent article is thus intended in which the quantity of salt added corresponds to 3% by weight (% wet) of the maximum weight of liquid that the article can absorb, for example 3 g of salt are added if the absorption capacity is 100 ml. Since urine normally has an inherent salt content of about 1% by weight, the final content is therefore about 4% by weight.

The tests shown in the examples were all carried out with maximum load (the greatest quantity of liquid that the product can absorb) of the products. This corresponds to the least favorable situation, i.e. the dilution of the salt is at its greatest. In real use situations, a maximum load is rarely reached before the product is changed, i.e. the salt concentration is higher and the effect better.

Weights for different products now follow here. Examples are also given of how the maximum absorption capacity is calculated. The quantity of salt that has to be added can in this way be easily calculated for the respective products. The dry weight for the core in a female panty liner is 1-3 g, for sanitary towels 3-15 g, for infants' diapers 20-50 g, and for incontinence products 10-120 g (for the simplest to the heaviest protector).

The maximum absorption weight for chemical fluff pulp is usually given as 6 ml/g and for superabsorbent polymers it is usually given as 25 ml/g. An incontinence product with 54 g (divided into two layers of 17 and 34 g) of chemical fluff pulp and 18.5 g of superabsorbent polymer will then have a maximum absorption capacity of 787 ml, i.e. a maximum absorption of 324 ml for the chemical fluff pulp, and 463 ml for superabsorbent polymer. If such an article is to have a quantity of added salt corresponding to 3% by weight (% wet) of the absorption capacity volume, this will be about 24 g of salt.

An incontinence product with 47 g (divided into two layers of 14 g and 33 g) of chemical fluff pulp and 6 g of superabsorbent polymer will have a maximum absorption capacity of 432 ml. 3% by weight (% wet) of added salt calculated on the basis of the maximum absorption capacity is then about 13 g of salt. These calculations can be carried out for various products by a person skilled in the art, taking account of the composition of the core and its maximum absorption capacity.

If the salt is placed in different zones in the product, the concentration may be higher in certain areas. When the product is not maximally loaded, the concentration is also higher. And when the concentration rises, the effect is better.

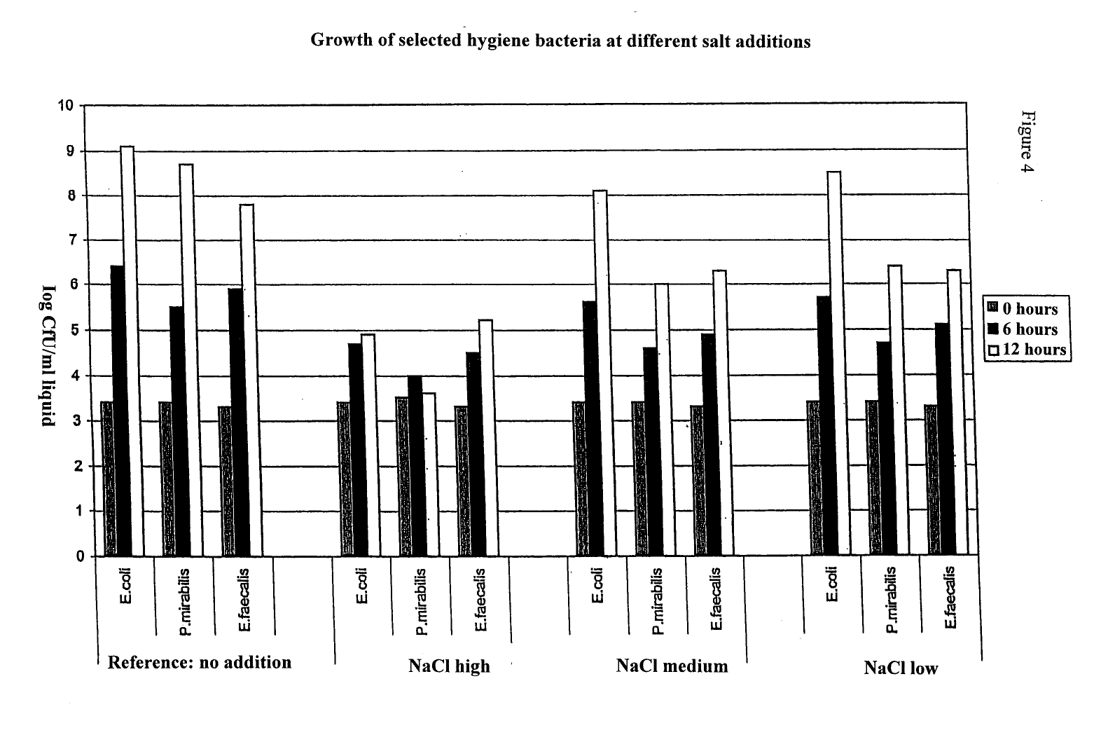

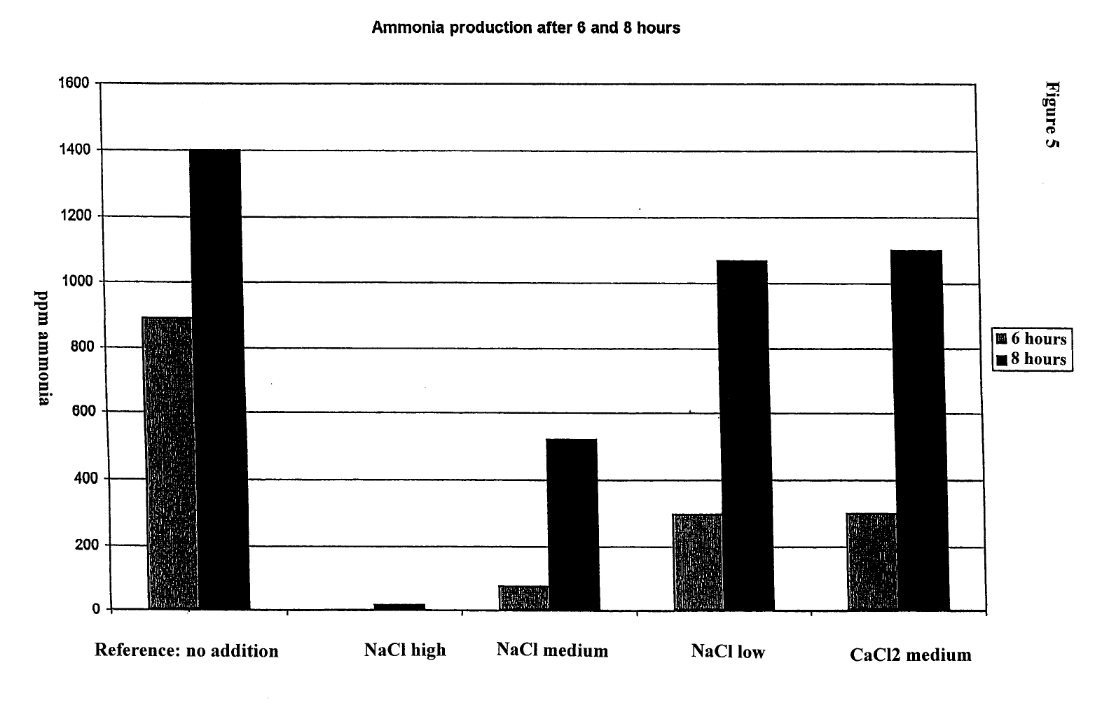

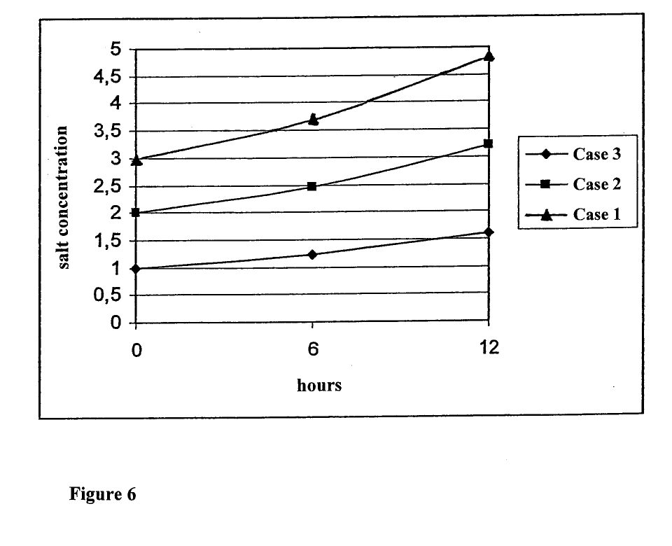

When vapor can be transported out through the vapor-permeable backing layer, the salt concentration will increase in the absorbent article. One example below shows that the salt concentration during one night for a night diaper with vapor-permeable backing layer can be increased from 2 to 3% by weight of salt, which can be seen in FIG. 6. This is an important change, since the quantity of bacteria and the development of ammonia decreases when the salt concentration is increased from 2 to 3% by weight, as can be seen in FIGS. 4 and 5.

E. coli, P. mirabilis and E. faecalis are chosen as relevant test bacteria in the examples. They are all examples of bacteria that one does not wish to have growing in large numbers in incontinence products, for example, during use. They can all cause urinary tract infections (UTI), for example. E. coli is often reported as the most common cause of UTI. Proteus is also urease-positive, which means that it can cleave urea to ammonia. Ammonia is an important cause of foul odors in used incontinence products.

For 3% added salt (% wet) (NaCl high in FIG. 4), the growth of all three test bacteria after 12 hours will lie below log 5.5. This is a great difference compared to the reference specimen, and a considerable hygiene improvement.

The vapor-permeable backing layer will also contribute to the water activity being kept down. In addition, it is possible to use vapor-permeable backing layers with greater vapor permeability, since bad odors from the absorbent article are reduced.

There now follow examples of different materials from which an absorbent article according to the invention can be made.

The cover layer can be made of a woven material, a nonwoven material, a polymer material such as perforated plastic films, porous foam, or reticulated foam. Suitable woven and nonwoven materials can include natural fibers (e.g. cellulose or cotton fibers), synthetic fibers (e.g. polymer fibers, such as polyesters, polypropylene or polyethylene) or a combination of natural and synthetic fibers. Nonwoven materials can be produced in a number of different ways, such as spunbond, carded, wet-laid, meltblown, hydroentangled, and combinations of the different methods.

The vapor-permeable backing layer is often composed of a plastic film, for example produced from polyethylene, polypropylene or a laminate of these. By addition of a filler, e.g. titanium dioxide, chalk or the like, the layer is made vapor-permeable. The layer can also be stretched. It is also possible to use a liquid-permeable material that is treated such that is becomes liquid-impermeable. Such treatments can involve the surface being partially coated with a liquid-impermeable glue, the surface being coated with a hydrophobic material, the liquid-permeable layer being laminated together with a liquid-tight material, an initially liquid-permeable material being hot-calendered such that the surface melts and the material thus becomes liquid-impermeable. The coating is not applied to the whole surface; instead, pores remain through which vapor can pass. A vapor-permeable, but liquid-impermeable material can also include liquid-impermeable, vapor-permeable textile materials or laminates including a number of textile layers or layers of film and textile material. Examples of substantially liquid-impermeable but vapor-permeable textile materials are hydrophobic nonwovens, porous plastic films, foamed material with filled cells, laminates composed of one or more layers of spunbond combined with meltblown layers, filled plastic film, waxed material layers and treated wadding.

The backing layer can have an adhesive attachment in the form of beads of adhesive, for example, on that side of the backing layer facing away from the cover layer, to enable it to be secured in panties, underpants or knickers. A release material may be applied on top of the adhesive in order to protect the adhesive when the product is not in use.

The absorbent core can also be composed of one or more layers of cellulose fibers, for example cellulose fluff pulp, airlaid, dry-defibered or compressed pulp. Other materials that can be used include, for example, absorbent nonwoven material, foam material, synthetic fibre material or peat. Apart from cellulose fibers or other absorbent materials, the absorbent core can also comprise superabsorbent materials, superabsorbent polymers, which are materials in the form of fibers, particles, granules, films or the like. Superabsorbent polymers are inorganic or organic materials which are capable of swelling in water and are insoluble in water and which have the capacity to absorb at least 20 times their own weight of an aqueous solution containing 0.9% by weight of sodium chloride. Organic materials that are suitable for use as superabsorbent polymers can include natural materials such as polysaccharides, polypeptides and the like, and also synthetic materials such as synthetic hydrogel polymers. Such hydrogel polymers can include, for example, polyacrylic acid, alkaline metal salts of polyacrylic acids, polyacrylamides, polyvinyl alcohol, polyacrylates, polyacrylamides, polyvinyl pyridines and the like. Other suitable polymers include hydrolyzed acrylonitrile-grafted starch, acrylic acid-grafted starch, and isobutylene maleic acid anhydride copolymers and mixtures thereof. The hydrogel polymers are preferably slightly cross-linked to ensure that the material remains essentially insoluble in water. Preferred superabsorbent materials can be surface cross-linked so that the external surface or shell of the superabsorbent particle, fiber, sphere, etc., has a higher cross-linking density than the inner part of the superabsorbent. The proportion of superabsorbents in an absorbent core can be between 10 and 90% by weight, or preferably between 30 and 70% by weight.

The absorbent core can comprise layers of different materials with different characteristics as regards their ability to receive liquid, their liquid distribution capacity and storage capacity. The absorbent core is in most cases extended in the longitudinal direction and can, for example, be rectangular, T-shaped or hourglass-shaped. An hourglass-shaped core is wider in the front and rear parts than in the crotch part, in order to provide effective absorption, at the same time as the design makes it easier for the product to be shaped close to and around the wearer, thus providing a better fit around the legs.

In addition, the absorbent article can include a transport layer between the cover layer and the absorbent core. The transport layer is a porous, flexible material and can comprise one or more of the following: airlaid, wadding, tissue, carded fiber web, superabsorbent particles or superabsorbent fibers. A transport layer has a high instantaneous capacity to receive liquid and is able to store liquid temporarily before it is absorbed by the underlying absorbent core. The transport layer can cover all or parts of the absorbent core.

The cover layer, the backing layer and any intermediate materials are sealed at the edges of the product, which can be done by thermal sealing, for example, or by some other conventional means.

The absorbent article can also comprise wings on its sides. It can also comprise elastic in order to provide better contact with the body when the product is being worn, and also to reduce leakage.

Salt has thus been found to function extremely well in inhibiting the growth of bacteria in absorbent articles. Absorbent articles often comprise superabsorbent materials, and it is known that salt has a negative impact on many superabsorbent polymers, although there do exist superabsorbent polymers that are insensitive to salt. A monovalent salt, however, has little impact on them, and the positive effect on inhibition of bacteria outweighs this consideration in these cases. A monovalent salt is advantageous if superabsorbent materials are used in the article. The absorbent articles with bacterial inhibition are easy to produce. The salt in itself is inexpensive and environmentally friendly. Together with the vapor-permeable backing layer, positive effects are achieved, as outlined above, and are intensified as the salt concentration increases during use of the absorbent article. At the same salt content and with the same frequency of changing the article, enhanced hygiene is achieved, and since the temperature drops in the article when vapor escapes through the backing layer, enhanced comfort is achieved and less growth of microorganisms. A breathable product which is comfortable for the user is obtained and, in addition, the risk of infection during use is reduced.

The invention will now be illustrated by the following examples.

### EXAMPLES

Test liquid 1 is used for pH measurements, bacterial growth measurements and ammonia measurements (referred to in method 2): Sterile, synthetic urine to which a growth medium for microorganisms has been added. The synthetic urine contains monovalent and divalent cations and anions and urea and has been produced in accordance with the information in Geigy, Scientific Tables, vol. 2, 8th ed., 1981, page 53. The growth medium for the microorganisms is based on the information of Hook and FSA medium for enterobacteria. The pH in this mixture is 6.6.

Test liquid 2 is used for water activity measurements:

### Recipe—Synthetic Urine

Magnesium sulfate 0.66 g/l (100 ml stock solution for 5 liters)

Potassium chloride 4.47 g/l (100 ml stock solution for 5 liters)

Sodium chloride 7.60 g/l (38.0 g for 5 liters)

Urea (carbamide) 18.00 g/l (90.0 g for 5 liters)

Potassium dihydrogen phosphate 3.54 g/l (100 ml stock solution for 5 liters)

Disodium hydrogen phosphate, anhydrous 0.745 g/l (100 ml stock solution for 5 liters)

Triton X-100, 0.1% strength 1 m00 g/l (5.0 g for 5 liters)

Deionized water to 1 l(5.0 l for 5 liters)

New coccine (dye) 10% strength 0.4 g/l (2.0 g for 5 liters)

### Principle

The four stock solutions are prepared first. Chemicals and stock solutions are mixed to give the solution for use.

### Implementation

Weighing of the chemicals and preparation of the test liquid require accuracy so that the solution for use will have the correct properties. Prepare the stock solutions according to the recipes. Mix the solution of chemicals and stock solutions in the sequence stated in the recipe.

### Stock Solutions (Storage Time 1 Month)

0.274 M magnesium sulfate: dissolve 33 g of magnesium sulfate in deionized water to give 1 l. (To 5 l of stock solution: 165 g MgSO₄)

2.998 M potassium chloride solution: dissolve 223.5 g of potassium chloride in deionized water to give 1 l. (To 5 l of stock solution: 1,117.5 g KCl)

1.301 M potassium dihydrogen phosphate solution: dissolve 177 g of potassium dihydrogen phosphate in deionized water to give 1 l. (To 5 l of stock solution: 885 g KH₂PO₄)

0.262 M sodium dihydrogen phosphate solution: dissolve 37.25 g of disodium hydrogen phosphate in deionized water to give 1 l. (To 5 l of stock solution: 186.25 Na₂HPO₄)

### Solution for Use (Storage Period 3 Weeks)

Fill flask to 60% with deionized water

Add the magnesium sulfate solution

Add the potassium chloride solution

Add the sodium chloride

When it is dissolved, add the urea

When it is dissolved, add the potassium dihydrogen phosphate solution

Add the disodium hydrogen phosphate solution

Add Triton X-100

Fill with deionized water to the exact quantity

### Physical Properties

The solution for use has the following values:

Surface energy (surface tension) 60±3 mN/m

Conductivity 23±2 mS

pH 6.0±0.5

Temperature 22±2° C.

### Method 1: Production of Absorbent Test Specimens for Testing

Absorbent test specimens were punched out from an absorbent core produced in a test plant. A standard method for mat-forming of a core was used during production of the core in the test plant. The absorbent test specimen included a homogeneous mixture of fluff pulp, 0.72 g Weyerhauser pulp (NB 416) and 0.48 g of superabsorbent polymer (SAP) (Degussa SXM9135). The absorbent core, was compressed to a bulk of around 8-10 cm³/g. The size of the punched-out test items was 5 cm in diameter, the weight was about 1.2 g.

### Method 2: Measurement of Ammonia Formation in Absorbent Cores

Absorbent test specimens were produced according to method 1. A bacterial suspension of Proteus mirabilis was cultured in nutrient solution at 30° C. overnight. The grafted cultures were diluted with test liquid 1 and the quantity of bacteria was determined. The final culture contained approximately 10⁵ organisms per ml of test liquid. The absorbent core was placed in a plastic tub and test liquid 1 containing bacteria was added to the absorbent core, after which the container was incubated at 35° C. for 4, 6 and 8 hours, and then the specimens were removed from the container using a hand pump and a so-called Drager tube. The ammonia content was obtained as a color change on a scale graduated in ppm or percent by volume.

### Method 3: Measurement of Bacterial Growth in Absorbent Cores

16 ml (corresponding to a maximum absorption capacity of a test specimen according to method 1) of test liquid 1 containing the bacteria were added to a test specimen placed in plastic tubs, and a lid was fitted on the tubs. The tubs were turned upside down and incubated in a warm cabinet at 35° C. After incubation for 0, 6 and 12 hours, the test samples were placed in a plastic bag with peptone water and the content was homogenized (agitated and worked up) in a stomacker for 3 minutes. The homogenate was diluted in dilution tubes with peptone water and a microbiological culture was spread on agar plates. Slanetz Bartley agar was used for E. faecalis, and Drigalski agar for E. coli and P. mirabilis. The specimens were incubated at 35° C. for 1-2 days before the colonies were counted and the log CFU/ml calculated. Control tests were also carried out with absorbent cores without NaCl.

### Method 4: Measurement of Water Activity

Water activity is measured as follows. The water activity meter is from Aqua Lab, Model Series 3 TE, Pentagon Devices Inc. (U.S. Pat. No. 5,816,704). A specimen, a solution with different additions of different salts in Elga-H₂O or synthetic urine, test liquid 2. was placed in a plastic cup. The quantity is not critical, but it is important that the bottom surface of the plastic cup is covered. The specimen is moved into the test chamber, which is closed, and the measurement commences. When the test value is stable, a green light flashes and the water activity can be read off digitally. The salts that were tested were: NaCl, KCl, NH₄Cl, KC₂H₃O₂ (K acetate), Na C₂H₃O₂ (Na acetate).

### Example 1

### Bacterial Growth on Addition of NaCl

Bacteria were cultured in nutrient broth and diluted to the desired concentration of about Log 3.3 in test liquid 1 (method 4). Absorbent test specimens were produced according to method 1. 29 (high), 22 (medium) and 12 (low) % by weight NaCl (% dry) were added to the core, corresponding to 3 (high), 2 (medium) and 1 (low) % by weight (% wet) in the wet product. This proportion also applies to the other examples. The bacterial growth was measured according to method 3.

The result is shown in FIG. 4, which clearly illustrates that the growth of all 3 test bacteria is lower after 6 and 12 hours, compared with the control tests, but also that the inhibition is better with a higher salt content.

### Example 2

### Development of Ammonia on Addition of NaCl or CaCl₂

Absorbent test specimens were produced according to method 1. 16 ml, maximum absorption capacity, of test liquid 1 containing bacteria were added to a test specimen. 29 (high), 22 (medium) and 12 (low) % by weight of NaCl (% dry) and 22 (medium) % by weight of CaCl₂ (% dry) were added to the core. The quantity of developed ammonia was measured according to method 2 after 6 and 8 hours. Control tests were also carried out using absorbent test specimens without NaCl or CaCl₂.

The results are shown in FIG. 5. The quantity of developed ammonia was lower for all the specimens with added salt compared to the control specimens. The lowest development of NH₃ was with the specimen containing 29% by weight of NaCl.

### Example 3

### Measurement of Salt Concentration in Combination with Vapor-Permeable Backing Layer

Calculations were based on a product from which a specimen was punched out, the specimen had a diameter of 5 cm and included a cover material of polypropylene nonwoven, superabsorbent particles (SAP) mixed with cellulose fluff and a backing layer of polyethylene Mocon 8000 g/m² and 24 h, which corresponds to 333 g/m² and h. A more realistic vapor permeability value was measured at 250 g/m² and h, using a vapometer (SWL 3 NO211 from Delfin Technologies Ltd.) The measurement was carried out on wet product for 1 h with the vapometer.

16 ml of test liquid 2 were added to the diaper specimen containing different amounts of added salt. The surface area of the diaper specimen was 1,963×10⁻³ m² and had an approximate value of 0.5 g/h evaporation. 0.48 g, 0.32 and 0.16 g of NaCl were added to the specimens 1, 2 and 3, respectively. The increase in the salt concentration is shown in FIG. 6.

For test 2, the salt concentration was changed from 2 to just over 3% by weight (wet %) for 12 hours, which can correspond to one night's use of a diaper. A salt concentration increase from 2 to 3% by weight (wet %) leads to a reduction in the development of ammonia, which can be seen in FIG. 5, and a reduced growth of bacteria, which can be seen in FIG. 4. For all the salt concentrations that were examined, the salt concentration increased when measured at 6 hours and at 12 hours.

### Example 4

### Measurement of Water Activity on 3% Strength Salt Solutions

The water activity was measured in Elga-H₂O (USF Elga, distilled water) and synthetic urine, test liquid 2. To show that additions of salt lead to a reduction in water activity, the water activity for different salts was measured in Elga water and test liquid 2. On measurement of the water activity in test liquid 2 with its inherent salt content, a water activity of 0.984 was measured. For Elga water, the water activity was measured to 0.999.3% by weight of various different salts were added to test liquid 2 and Elga water and the water activity was measured according to method 4.

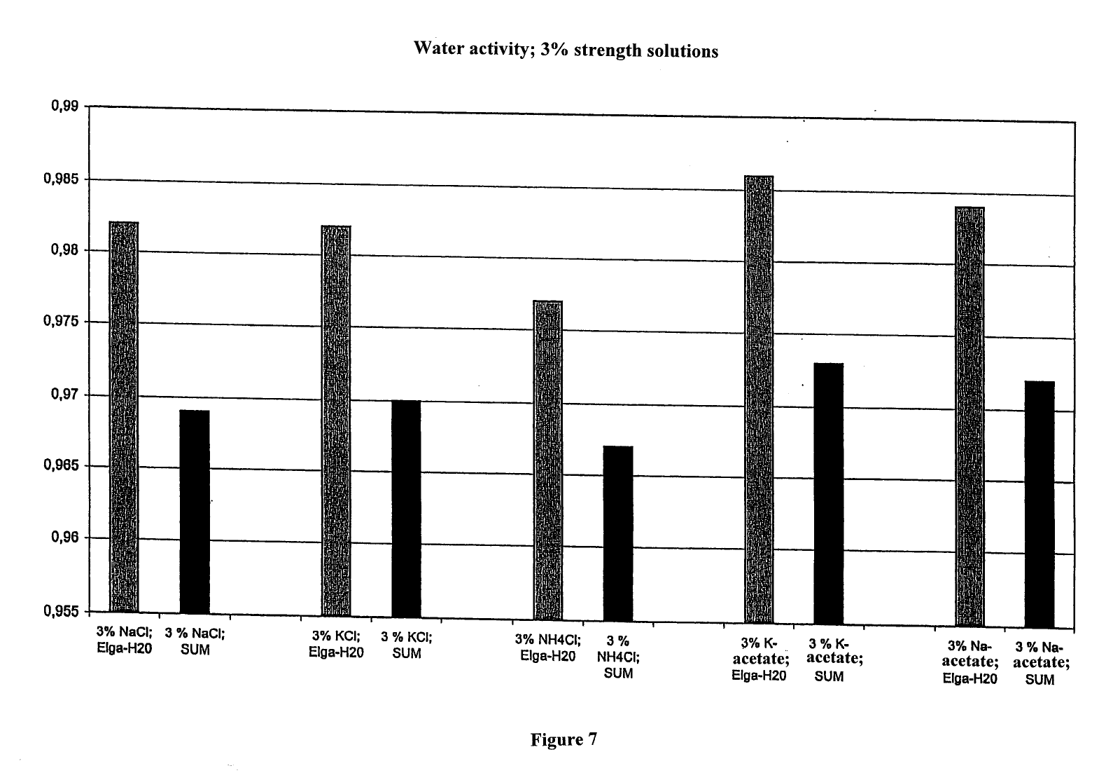

Clear decreases in water activity are shown in FIG. 7. The water activity after addition of 3% salt in test liquid 2 is below 0.975.

### Example 5

### Water Activity at Different Concentrations of NaCl, Na acetate, NH₄Cl

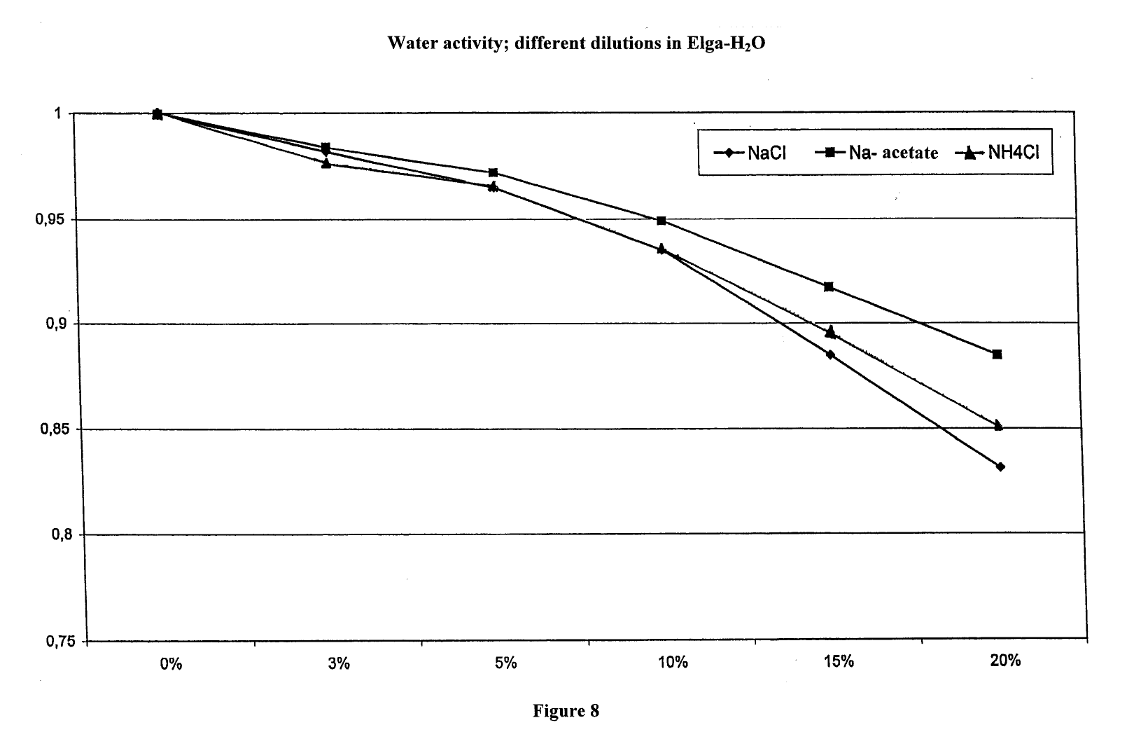

The water activity was measured according to method 4, but with different concentrations of salt in Elga-H₂O. FIG. 8 shows clearly how the water activity drops as the concentrations of salt increase.

1. An absorbent article, such as a diaper, diaper pants, sanitary towel or incontinence protector, with a vapor-permeable backing layer, comprising a liquid-permeable cover layer intended to be directed toward the user during use, a liquid-impermeable but vapor-permeable backing layer intended to be directed away from the user during use, and an absorbent core between the cover layer and the backing layer, said article including a longitudinal direction, a transverse direction, two substantially longitudinal side edges, a substantially transverse front edge, a substantially transverse rear edge, a substantially longitudinal midline, and a front part and a rear part on each side of a substantially transverse center line, said midline and center line intersecting one another at a point of intersection, wherein the absorbent article comprises at least one monovalent salt of the form X⁺Y⁻ in a quantity of 1-75% by weight calculated on the basis of the weight of the core, wherein X⁺ is chosen from among Na⁺, K⁺, NH₄⁺, and Y⁻ is chosen from among Cl⁻, C₂H₃O₂⁻ and C₃H₅O₂⁻.

2. (canceled)

3. The absorbent article as claimed in claim 1, wherein the salt is distributed within a central area around the point of intersection.

4. The absorbent article as claimed in claim 1, wherein the area extends at least 1.5 cm from the center line in the longitudinal direction and at least 1.5 cm from the midline in the transverse direction.

5. The absorbent article as claimed in claim 1, wherein the vapor-permeable backing layer has a vapor permeability of at least 500 g/m2 and 24 h.

6. The absorbent article as claimed in claim 1, wherein the salt is distributed within the absorbent core.

7. The absorbent article as claimed in claim 1, wherein the salt is distributed on top of the absorbent core.

8. The absorbent article as claimed in claim 1, wherein the absorbent article further comprises a spreader layer.

9. The absorbent article as claimed in claim 8, wherein the spreader layer is arranged between the cover layer and the absorbent core.

10. The absorbent article as claimed in claim 8, wherein the spreader layer is arranged between the absorbent core and the backing layer.

11. The absorbent article as claimed in claim 8, wherein the spreader layer is arranged within the absorbent core.

12. The absorbent article as claimed in claim 1, wherein the salt has a particle size of 50 to 1500 μm.

13. The absorbent article as claimed in claim 1, wherein the salt is added in the form of flakes, and the flakes have a width dimension of up to 1 cm.

14. The absorbent article as claimed in claim 1, wherein the salt is added as pure salt.

15. The absorbent article as claimed in any of the claim 1, wherein the salt is added in a quantity such that the water activity in the absorbent article is under 0.98 during wetting.

16. The absorbent article as claimed in claim 1, wherein the salt is added in a quantity such that the concentration of each of E. coli, P. mirabilis and E. faecalis can be maintained below 6.5 Log CFU/ml in the article after 10 hours of wetting with synthetic urine.
